(19) United States
(12) Patent Application Publication
(10) Pub. No.: US 2004/0035793 A1
(43) Pub. Date: Feb. 26, 2004

(54) Methods, systems, and kits for analysis of polynucleotides

(75) Inventors: Benjamin L. Legendre JR., Omaha, NE (US); Joseph G. Rudolph III, Silver Spring, MD (US); Michael A. Marino, Frederick, MD (US)

Correspondence Address: KEITH JOHNSON, ESQ., TRANSGENOMIC, INC., 12325 EMMETT STREET, OMAHA, NE, 68164, US

(73) Assignee: Transgenomic, Inc., San Jose, CA

(21) Appl. No.: 10/288,406

(22) Filed: Nov. 4, 2002

Related U.S. Application Data
(60) Provisional application No. 60/338,627, filed on Nov. 5, 2001.
(60) Provisional application No. 60/338,041, filed on Dec. 4, 2001.
(60) Provisional application No. 60/370,749, filed on Apr. 5, 2002.

(51) Int. Cl. B01D 15/08

(52) U.S. Cl. 210/656; 210/198.2; 436/161; 530/417

(57) ABSTRACT

Methods, systems, compositions and kits for improved detection of polynucleotides. In one aspect, there is provided a method for separating polynucleotides (such as DNA or RNA) using a liquid chromatographic separation device (such as a reverse phase column or an ion exchange column), contacting eluted polynucleotides with intercalating dye, and detecting (such as by fluorescence detection) dye bound to the eluted polynucleotides. The invention preferably uses a post-column reactor, such as a mixing tee, downstream of the separation column. Sensitivity of mutation detection by denaturing high performance liquid chromatography (DHPLC) is enhanced.

### CROSS-REFERENCE TO RELATED APPLICATIONS

This application is a regular U.S. patent application under 35 U.S.C. §111 (a) and 37 U.S.C. §1.53(b) and claims priority from the following co-pending, commonly assigned provisional applications, each filed under 35 U.S.C. §111 (b): U.S. Patent Application No. each of which is incorporated herein by reference: No. 60/338,627, filed Nov. 5, 2001; No. 60/338,041, filed Dec. 4, 2001; and No. 60/370,749, filed Apr. 5, 2002.

### FIELD OF THE INVENTION

The invention generally relates to the field of polynucleotide separations and more specifically concerns improvements in the detection of polynucleotides which have been subjected to separation techniques.

### BACKGROUND OF THE INVENTION

Separations of polynucleotides such as DNA have been traditionally performed using slab gel electrophoresis or capillary electrophoresis. However, liquid chromatographic separations of polynucleotides are becoming more important because of the ability to automate the analysis and to collect fractions after they have been separated.

High quality materials for DNA separations have been based on polymeric substrates (as described in U.S. Pat. Nos. 5,585,236 and 6,066,258) and on silica-based reverse phase column materials (as described in U.S. Pat. Nos. 6,056,877 and 6,156,206). Detection of eluted polynucleotides typically utilizes ultraviolet detection for which the limit of detection is about 5 to 10% above background. Polynucleotides subjected to liquid chromatographic analysis, and other separation techniques, often include polymerase chain reaction (PCR) products. PCR amplification is routinely performed using covalently tagged PCR primers to incorporate a detectable moiety (e.g. a fluorescent label) into the amplification product in order to increase the sensitivity of detection. However, fluorescent tags and other covalent tags add to the cost of the PCR method. Such tags are usually hydrophobic and can significantly alter the chromatographic retention time of a fragment. There is a need for increased sensitivity in the detection of polynucleotides that have been subjected to various separation methods. There is also a need for methods that do not require the use of covalently tagged PCR primers.

### SUMMARY OF THE INVENTION

In one aspect, the invention concerns a method for analyzing one or more polynucleotides in a mixture. In one embodiment, the method includes (a) separating the polynucleotides using a liquid chromatographic separation device wherein the polynucleotides are eluted from the device; (b) contacting eluted polynucleotides with intercalating dye such that the dye binds to the eluted polynucleotides; and (c) detecting the dye bound to the eluted polynucleotides. The device preferably includes a separation column, such as a reverse phase column or an ion exchange column. The contacting preferably includes flowing the mixture through a post-column reactor, such as a mixing tee or a mixing cross. Other examples of suitable reactors include a “Y” union; a multiport union having one outlet and greater that two inlets; a multiple inlet mixing valve; and a switching valve. A preferred dye is one that exhibits fluorescence only when binding with a polynucleotide. A more preferred dye is one that exhibits fluorescence only when intercalated with a polynucleotide. The method can include heating the reagent such that the column and the reagent are retained at essentially the same temperature. The polynucleotides can include DNA or RNA, single-stranded or double-stranded molecules. The polynucleotides can include homoduplex and heteroduplex molecules. The dye preferably is a nucleic acid stain. The dye can be selected from SYBR Green I, SYBR Green II, and mixtures thereof. Another example if SYBR Gold. The method can include analyzing the polynucleotide product of step (b) by mass spectral analysis. In another aspect, the invention concerns a composition comprising polynucleotide product resulting from the above method.

In a further aspect the invention concerns a method for analyzing one or more polynucleotides. In one embodiment, the method includes (a) a step for separating the polynucleotides using a liquid chromatographic separation device wherein the polynucleotides are eluted from the device; (b) a step for contacting eluted polynucleotides with intercalating dye such that the dye binds to the eluted polynucleotides; and (c) a step for detecting the dye bound to the eluted polynucleotides.

In another aspect, the invention provides an apparatus for analyzing polynucleotides. In one embodiment, the apparatus includes (a) a liquid chromatographic separation column capable of separating polynucleotides by ion-pair reverse-phase high performance liquid chromatography; and (b) a reactor for mixing intercalating dye reagent with polynucleotides eluted from the column. The column can be a reverse phase separation column or an ion exchange column. The apparatus can further include a detector, such as a fluorescence detector, capable of detecting the dye bound to polynucleotides. The reactor can be a mixing tee or mixing cross. Other examples of suitable reactors include a “Y” union; a multiport union having one outlet and greater that two inlets; a multiple inlet mixing valve; and a switching valve. The apparatus can further include a heater for heating the dye reagent to essentially the same temperature as the column. The apparatus can include an ultraviolet detector. The apparatus can also include a mass spectrometer operatively coupled to the separation column. In other embodiments, the apparatus can include (c) conduit connected to the end of the column for conducting mobile phase eluting from the column, the reactor connected to the tubing; (d) a reservoir containing the intercalating dye reagent including conduit for operatively connecting the reservoir to the reactor; and (e) a pump for pumping the dye reagent into the reactor such that the dye reagent mixes with the mobile phase.

In an additional aspect, the invention provides an apparatus for analyzing polynucleotides. In a preferred embodiment, the apparatus includes (a) a chromatographic means for separating one or more polynucleotides; and (b) means for mixing intercalating dye with polynucleotides eluted from the device. Examples of suitable chromatographic means include a reverse phase separation column and an ion exchange column. The apparatus preferably further includes means for detecting intercalating dye bound to polynucleotides eluted from the chromatographic means. The detector means is preferably a fluorescence detector. The means for mixing is preferably a post-column reactor, such as a mixing tee adapted to mix the intercalating dye and polynucleotides eluted from the means for separating.

In another aspect, the invention provides a chromatographic method for separating heteroduplex and homoduplex DNA molecules in a mixture. The method includes applying the mixture to a stationary reverse phase support; eluting the heteroduplex and homoduplex molecules of the mixture with a mobile phase containing an ion-pairing reagent and an organic solvent, where the eluting is carried out under conditions effective to at least partially denature the heteroduplexes and where the eluting results in the separation of the heteroduplexes from the homoduplexes; contacting the heteroduplex and homoduplex molecules with intercalating dye reagent after the eluting; and detecting the dye bound to the heteroduplex and homoduplex molecules. In the method, the stationary support can be composed of an alkylated base material, the base material selected from the group consisting of silica, alumina, zirconia, polystyrene, polyacrylamide, and styrene-divinyl copolymers. The mobile phase preferably contains an ion-pairing agent selected from the group consisting of lower alkyl primary, secondary, and tertiary amines, lower trialkylammonium salts and lower quaternary ammonium salts. A preferred mobile phase includes triethylammoniumacetate. The mobile phase can contain an organic solvent selected from the group consisting of methanol, ethanol, acetonitrile, ethyl acetate, and 2-propanol. An example of a suitable mobile phase contains less than about 40% by volume of the organic solvent. The method can include heating the reagent to essentially the same temperature as the heteroduplex and homoduplex molecules under the conditions. The dye can be selected from the group consisting of SYBR Green I stain, SYBR Green II stain, and mixtures thereof. Other examples of suitable dye are SYBR Gold and PicoGreen.

In yet another aspect, the invention concerns a kit for detecting polynucleotides. A kit can include one or more of the following: intercalating dye reagent; a reactor for mixing intercalating dye reagent with mobile phase eluting from a liquid chromatography column; a liquid chromatography column; a pump for pumping a solution of the dye into the reactor; a detector for detecting the dye; conduit for connecting the reactor to the column; in a separate container, a standard mixture of polynucleotides (e.g. a standard mixture of polynucleotides of double stranded polynucleotides, such as DNA); in a separate container an intercalating dye, such as SYBR Green I stain, SYBR Green II stain, or a mixture thereof; in a separate containers, SYBR Gold nucleic acid stain or PicoGreen.

In still another aspect the invention provides an apparatus for analyzing polynucleotides including: (a) means for chromatographic separation wherein one or more polynucleotides can be applied to the means for chromatographic separation and can be eluted from the means for chromatographic separation; (b) means for adding or mixing intercalating dye with polynucleotides eluted from the means for chromatographic separation; and (c) means for detecting intercalating dye bound to polynucleotides eluted from the means for chromatographic separation. The means for chromatographic separation can comprise a reverse phase liquid chromatography column or an ion exchange column. The means for adding or mixing can comprise a mixing tee, a liquid flow-through reactor, or a hollow fiber membrane.

In yet another aspect, the invention provides an apparatus for analyzing polynucleotides including (i) a liquid chromatographic column having an outlet; (ii) a mixing tee having a first inlet, a second inlet, and an outlet with the first inlet in fluid communication with the outlet of the chromatographic column; (iii) wherein the second inlet is in fluid communication with a fluid source, wherein the fluid source comprises an intercalating dye reagent. The apparatus preferably includes a heater (e.g. a thermostatically controlled heater) for heating the dye reagent.

In another aspect, the invention includes a liquid chromatographic apparatus such as a silica based chromatographic column means or a polymeric based chromatographic column means, a reservoir of mobile phase in fluid communication with the column means, a chromatographic pump means to add the mobile phase to the column means, whereby the sample comprising a mixture of at least one polynucleotide is eluted through the column means, and component species of the mixture appear in chromatographically displaced form in the effluent of the chromatographic column means, and further including a post-column reactor means through which the effluent of the chromatographic column means is fed to a liquid chromatographic detector, a medium comprising intercalating dye reagent, the reactor means being in operative contact or communication with the medium for transfer of the reagent into the effluent of the chromatographic column means. Example of a suitable post-column reactor means a hollow fiber membrane, a mixing tee, and a mixing cross. The apparatus can include a pump for pumping the medium into effluent from the chromatographic column means. Examples of a suitable pump include a syringe, a peristaltic pump, or an HPLC pump.

In an additional aspect, the invention provides a chromatographic apparatus for separating polynucleotides, the apparatus including: a reverse phase separation column, a post-column reactor located downstream of the column, a medium containing intercalating dye, wherein the reactor is adapted to mix mobile phase eluted from the column with the medium, a fluorescence detector downstream of the reactor for detecting intercalating dye bound to polynucleotides. The column can include a silica stationary support or a polymeric stationary support.

In a further aspect, the invention concerns a method for analyzing one or more polynucleotides. The method preferably includes (a) separating the polynucleotides using capillary electrophoresis; (b) contacting the polynucleotides with intercalating dye; and (c) detecting the dye bound to the polynucleotides.

### BRIEF DESCRIPTION OF THE DRAWINGS

FIG. 1 is a schematic illustration of an embodiment of a chromatographic system of the invention.

FIG. 2 is a schematic illustration of an embodiment of a post-column reactor.

FIG. 3 exemplifies DHPLC analysis of a mixture of homoduplex and heteroduplex molecules.

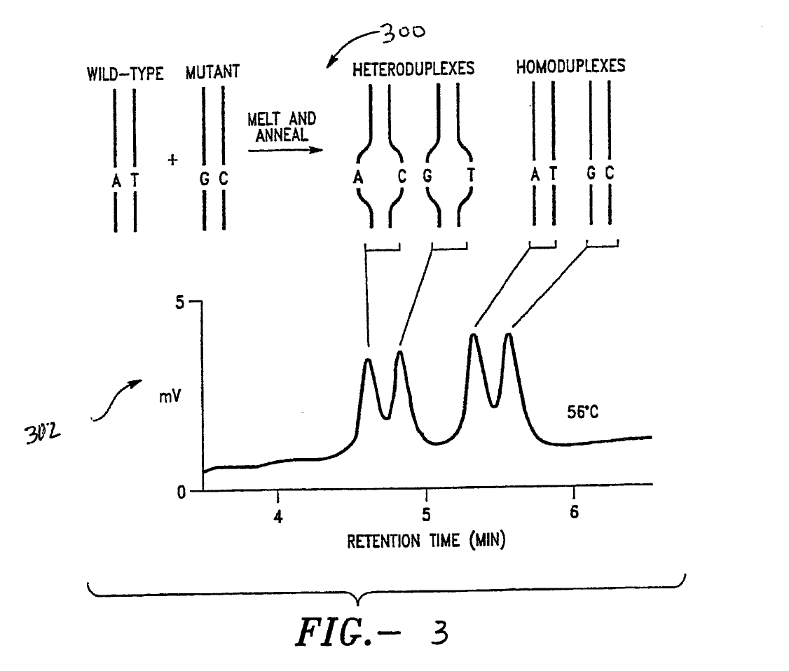

FIG. 4 illustrates the IP-RP-HPLC separation of a mixture of polynucleotides with UV detection (FIG. 4A) and fluorescence detection (FIG. 4B).

FIG. 5 illustrates the IP-RP-HPLC separation of a first dilution of the mixture of polynucleotides of FIG. 4 with UV detection (FIG. 5A) and fluorescence detection (FIG. 5B).

FIG. 6 illustrates the IP-RP-HPLC separation of another dilution of the mixture of polynucleotides from FIG. 4 with UV detection (FIG. 6A) and fluorescence detection (FIG. 6B).

FIG. 7 illustrates the IP-RP-HPLC separation of homoduplex and heteroduplex molecules at a non-denaturing temperature with UV detection (FIG. 7A) and fluorescence detection (FIG. 7B).

FIG. 8 illustrates the DHPLC analysis of homoduplex and heteroduplex molecules with UV detection (FIG. 8A) and fluorescence detection (FIG. 8B).

### DETAILED DESCRIPTION OF THE INVENTION

In a general aspect, the invention concerns methods, compositions, systems and kits for enhancing the detection of polynucleotides that have been subjected to a separation technique, such as liquid chromatographic separation. The invention is based in part on Applicants' observation that polynucleotides, as eluted from a separation column, can be mixed with various binding agents in order to enhance detection of the polynucleotides. Applicants have surprisingly discovered that the mixing of intercalating dyes (as described hereinbelow) with the effluent from a separation column effected a marked increase in the sensitivity of detection.

The term “polynucleotide” is defined to include a linear polymer containing an indefinite number of nucleotides, linked from one ribose (or deoxyribose) to another via phosphoric residues. The present invention can be used in the separation of RNA or of double- or single-stranded DNA. For purposes of simplifying the description of the invention, and not by way of limitation, the separation of double-stranded DNA will primarily be described herein, it being understood that all polynucleotides are intended to be included within the scope of this invention.

A variety of methods are known for the separation of polynucleotides, including liquid chromatographic techniques such as ion exchange chromatography and ion-pair liquid chromatography. The use of ion exchange chromatography is disclosed, for example, in U.S. patent application Ser. No. 09/756,070 filed Jan. 6, 2001 and WO 01/27331. For purposes of clarity and not by way of limitation, ion-pair reverse-phase high performance liquid chromatography (IP-RP-HPLC) will primarily be described herein.

A preferred IP-RP-HPLC system provides automated options for sample selection, mobile phase gradient selection and control, column and mobile phase temperature control, and fraction collection.

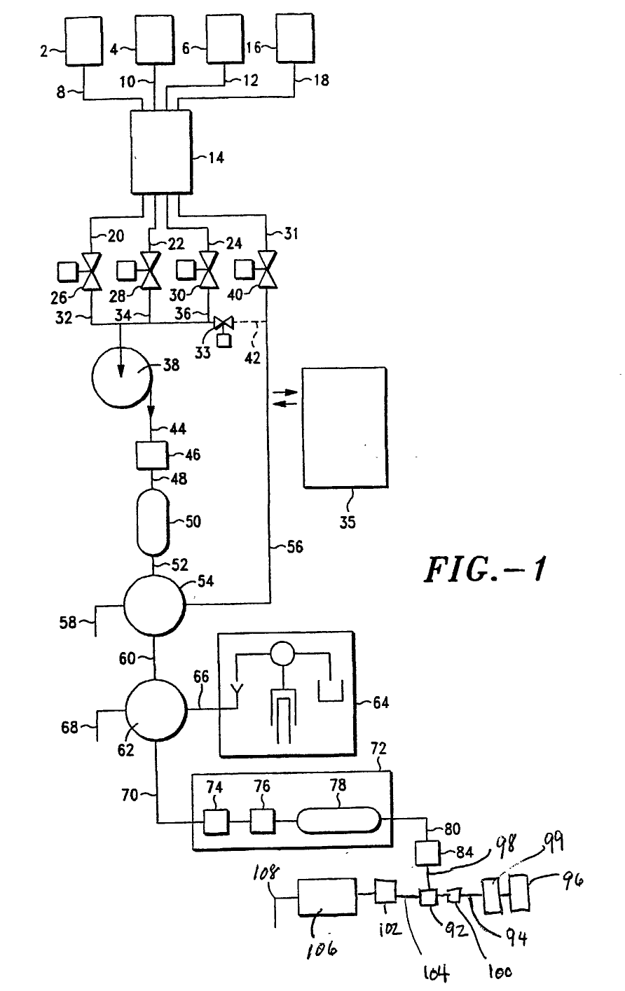

FIG. 1 is a schematic layout of the system in accordance with one embodiment of the IP-RP-HPLC system. A plurality of containers can be used as reservoirs for solutions, such as solvents, counterions, and other solutions, which make up the mobile phase. For example, container 2 can contain an aqueous component of a mobile phase such as an aqueous solution of counterion agent (e.g., triethylammonium acetate (TEAA)), and container 4 can contain an aqueous solution of counterion agent plus organic (driving) solvent (e.g., TEAA plus acetonitrile). An auxiliary liquid (e.g., a co-solvent) can be held in container 6. These solutions are mixed to achieve a selected concentration of organic solvent in the mobile phase during a separation. Other examples of these solutions are provided in the Examples herein and in the commonly assigned patent indicated hereinabove. The containers have respective transport tubing such as counterion solution transport tubing 8, solvent solution transport tubing 10, and auxiliary liquid transport tubing 12 communicating therewith, and leading to degasser 14.

The degasser 14 removes dissolved gases from the liquids. An example of a suitable degasser is the Degassit Model 6324. Removal of dissolved oxygen is particularly important because its presence increases the risk of oxidizing ferrous or other oxidizable metals in the system components and thus introducing the corresponding cations into the mobile phase liquid.

Column cleaning solution is contained in cleaning solution container 16 which likewise has a cleaning solution transport conduit 18 communicating therewith leading to the degasser 14. In this embodiment, the cleaning solution can flow by gravity pressure if the container 16 is elevated above the degasser and injection valve 54.

The system of the invention incorporates conventional mobile phase flow control means which controls flow of solvent solution and aqueous components of a mobile phase. In one embodiment, the mobile phase flow control means comprises a set of flow control valves, each with automatic opening controls under computer control as described hereinbelow. In another embodiment the mobile phase flow control means comprises a set of pumps, the flow setting of which are responsive to computer control as described hereinbelow.

The system illustrated in FIG. 1 utilizes one embodiment of a mobile phase flow control means which includes a set of flow control valves. Degassed counterion solution conduit 20, degassed solvent solution conduit 22, and degassed auxiliary liquid conduit 24 leading from the degasser 14 communicate with respective aqueous component proportioning valve 26, solvent solution proportioning valve 28, and auxiliary liquid proportioning valve 30. The settings for these proportioning valves are set and changed by valve operators such as stepper motors associated therewith, and these valve operators respond to establish a desired set of settings in response to commands from the mobile phase flow control software module described in greater detail hereinbelow. The flow control valves 26, 28, and 30 comprise an embodiment of a mobile phase flow control means which controls the flow of solvent solution and other components of the mobile phase. The settings for these valves control the ratio of liquids (co-solvents, solvent solution, etc.) through the injector valve and the separation column. Conduits 32, 34, and 36 lead from respective proportioning valves 26, 28 and 30 to the intake of pump 38.

The cleaning solution transport conduit 31 leads to a cleaning solution valve 40. An optional cleaning solution conduit 42 leads from the valve 40 and communicates with the inlet of pump 38. Valve 33 controls flow through conduit 42.

The openings of valves 26, 28 and 30 accurately set the relative ratios of the organic solvent, and other components, within the mobile phase, a most important part of this system because polynucleotide separation by IP-RP-HPLC is a function of solvent concentration. As will be described in regard to the various polynucleotide separation processes, the slope of the organic solvent gradient as a function of time is changed during the separation process, and the most critical phase may require a very precise gradient. The settings of the valves 26, 28 and 30 are established by conventional valve actuators which can be remotely set by signals to a conventional valve control device.

In a preferred embodiment, the separation system is under computer control as represented at 35. The computer includes Instrument Control Software which provides computer controlled instructions for establishing the settings of valves 26, 28 and 30 to precise flow values at appropriate times during the operation of the system.

In a similar manner, the Instrument Control Software of the instant invention provides computer controlled instructions to establish the operational parameters of the pump 38, such as the off/on status of the pump and the pressure or flow rate settings of the pump.

Pump outflow conduit 44 communicates with the in-line mixer 46, directing the liquid flow through the mixer 46 for thorough mixing of the components. Mixed liquid outflow conduit 48 communicates with optional guard column 50 to treat the mixed liquid to remove multivalent metal cations and other contaminants which would interfere with the separation of polynucleotide molecules. Guard column 50 can contain a cation exchange resin in sodium or hydrogen form for removal of multivalent metal cations by conventional ion exchange. Conduit 52 communicates with the outlet of the guard column and an inlet port of a cleaning solution injector valve 54. Cleaning solution supply conduit 56 connects valve 40 with the cleaning solution injector valve 54, and waste outlet conduit 58 leads to waste. Conduit 60 leads from valve 54 to the sample injection valve 62.

Sample aliquot selector 64 communicates with injector valve 62 through sample conduit 66. Waste conduit 68 leads from the injector valve and removes waste liquids.

In the injector valve 62, the sample is introduced into a stream of solvent and carrier liquid passing through the valve from conduit 60. Sample conduit 70 communicates with an outlet port of injector valve 62 and with the column prefilter 74 in the air bath oven 72. The capillary tubing coil 76 communicates with the prefilter 74 and the inlet of chromatography column 78. The extended length of the capillary coil 76 allows ample heat to pass from the heated oven air into the liquid passing through the coil, bringing the liquid within ±0.05° C. of a selected temperature. The oven 72 establishes this temperature uniformity in the prefilter 74, coil 76, and chromatography column 78.

The separation column 78 is packed with beads having a unique separation surface which effects separation of polynucleotide molecules in the presence of a counterion by the IP-RP-HPLC process. The separation process and details about the column and beads are described in detail hereinbelow. A stream of mobile phase containing separated polynucleotide molecules passes from the chromatography column 78 through conduit 80.

Conduit 80 communicates with an optional detector 84. The detector can be a conventional UV absorbance device which measures the UV absorbance of the polynucleotide fragment structures in the liquid mobile phase. The absorbance is a function of the concentration of the polynucleotide fragments in the liquid being tested.

In the above description, the liquid flow system is described as a series of conduits. The conduits are capillary tubing selected to avoid introduction of multivalent cations into the liquids. The preferred capillary tubing materials are titanium and PEEK. The other components of the system are preferably made of titanium or PEEK or have the surfaces exposed to the liquid coated with PEEK to protect them from oxidation and prevent the introduction of multivalent cations into the liquid. Stainless steel can also be used but is preferably treated to remove all oxidized surface materials and the solutions contacting the stainless steel surfaces are free of dissolved oxygen.

In preferred embodiments of the present invention, the system includes a post-column reactor 92 which is positioned downstream of the column 78. The reactor communicates via conduit 94 with reservoir 96 and with mobile phase eluting from the column via conduit 98. As will be described hereinbelow, reservoir 94 can contain a solution containing intercalating dye. A pump 99 can be used to achieve flow of solution from reservoir 94. An optional heating device 100 can be used to pre-heat fluid from reservoir 96 prior to reaching reactor 92.

A detector 102, such as a fluorescence detector, is positioned downstream of the reactor 92 and communicates with reactor 92 via conduit 104. The electrical output from the detector preferably is converted to a digital form by an A/D converter and recorded in standard digital format to a digital storage device such as a disk drive in computer 35.

Then, the mobile phase passes to the automated fraction collector 106 where selected portions of the mobile phase fractions can be collected in vials for later processing or analysis. Uncollected fractions are removed through conduit 108.

One aspect of the present invention concerns a post-column reactor (i.e. mixing device) for use in detecting polynucleotides after chromatographic separation. One or more post-column reactors can be positioned downstream of separation column as shown in FIG. 1. One embodiment of such a reactor is a conventional mixing tee. Mixing tees and mixing crosses are available commercially (e.g. Upchurch Scientific) and are readily adapted for use in chromatography systems. Examples of suitable reactors include: a mixing tee as described in U.S. Pat. No. 6,100,522 and as available commercially (e.g. part no. P-632 Upchurch); a mixing cross (part no. P-634, Upchurch); a “Y” union; a multiport union having one outlet and greater that two inlets; a multiple inlet mixing valve (part no. 080T-3-12-32-5, BioChem Valve Corporation); and a switching valve (part no. V-100T, Upchurch). Preferably the device is constructed to have inert inner surfaces (e.g. Teflon of Tefzel (ETFE)).

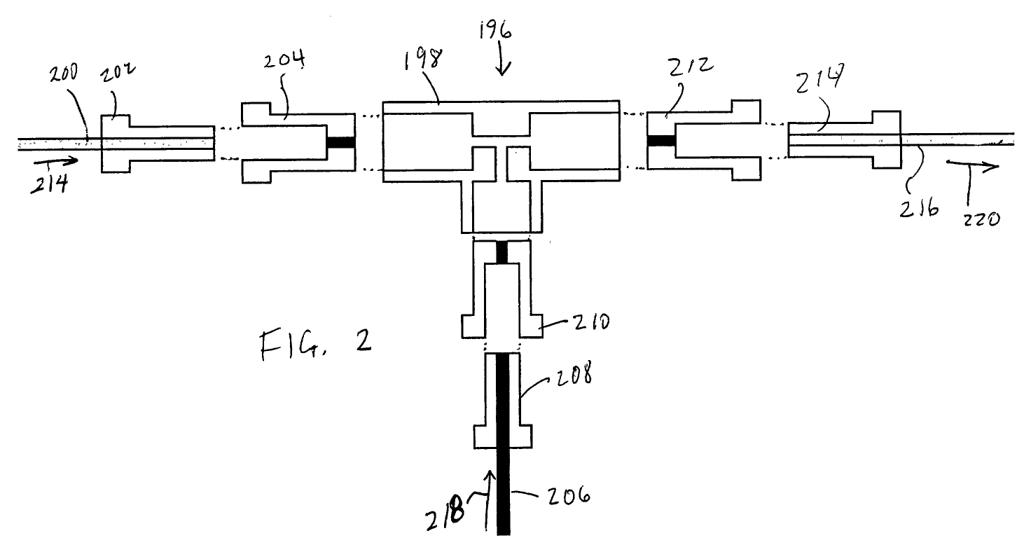

An embodiment of a suitable reactor in the present invention is the conventional mixing tee apparatus 196 shown in FIG. 2. Conduit 200 leads from a separation column and is retained within adaptor 202. Adaptor 202 threadably engages inlet valve 204, which threadably engages tee junction 198. Conduit 206, leading from a reservoir of dye reagent (not shown), is held within adaptor 208 which engages inlet valve 210. Valve 210 engages tee junction 198 as shown. Conduit 216 leads away from the mixing tee and toward a detector (e.g. a fluorescence detector). Conduit 216 is held within adaptor 214 which is engaged within outlet valve 212. Valve 212 engages tee junction 198 as shown. Mobile phase enters the device in the direction of arrow 214, dye reagent enters the tee device in the direction of arrow 218. Valves 204, 210 and 212 are preferably one-way valves, such as check valves. After mixing, the fluid flows in the direction of arrow 220.

Another suitable reactor for introducing a dye reagent includes a hollow fiber membrane such as described in U.S. Pat. Nos. 4,448,691 and 4,451,374.

The dye reagent is preferably dissolved in an aqueous solution. The concentration of the dye will be dependent on the fluorescent stain selected. The concentration of the dye can be between about 0.001 μM and 1M. The solution can include buffering agents, various solubilization or stabilization agents.

A pump can be used to provide flow of intercalating dye into the reactor (FIG. 1). An example is a conventional HPLC pump. Pulse dampening devices, such as described in U.S. Pat. No. 6,281,019, can be used in conjunction with a pump used with a post-column reactor as described herein. An example of a preferred pump is a SSI Series I reciprocating, single piston pump (Scientific Systems, Inc., State College, Pa.). The preferred flow rate range of the pump is 0.01 to 10.00 ml/min. The pump is preferably operated no less than 5000 psi backpressure and more preferably at no less than about 1000 psi back pressure. Applicants have found that they were able to adjust the back pressure by inserting a length of capillary tubing (i.e. a back pressure coil) between the pump and the reactor. Forcing the solution to go through the coil provided the preferred back pressure. The pressure could be “tuned” for a variety of flow rates by changing the length of the back pressure coil. In one embodiment, a 3 foot coil of PEEK tubing (75 μm ID) (SSI) was used.

In preferred embodiments of the instant invention, the intercalation dye solution is pre-heated prior to contact with mobile phase that is eluted from the separation column. For example, the solution can be heated to a temperature in the range of about 25° C. to about 70° C. Preferably the dye solution is heated to a temperature that is essentially the same as the column temperature. In one embodiment, the entire reservoir containing the dye solution is heated. In a preferred embodiment, a coil of capillary conduit (e.g. Teflon tubing) extending from the reservoir, is heated to provide pre-heating of dye reagent prior to introduction into the mixing device. The coil can be heated within the same column heater used for the separation column (FIG. 1), or can be a different heater. Examples of preferred heater devices are described in U.S. Pat. No. 6,103,122.

In the practice of the present invention, the intercalating dye-polynucleotide complex is detected using a fluorescence detector. Suitable detectors are available commercially (e.g. from Hewlett Packard (model 1046), Hitachi (model L7450), Gilson (model 121), WATERS (model 120), Bio-Rad (model 1700), and Beckman (model 6300A)). A preferred fluorescence detection device is a laser (e.g. argon laser) induced excitation source. A xenon arc lamp is less preferred as an excitation source.

In an important aspect, the invention provides a method for enhancing the detection of a polynucleotide separated by ion-pair reverse-phase high performance liquid chromatography, including (a) applying the polynucleotide to a separation medium having a non-polar surface, (b) eluting the polynucleotide from the surface with a mobile phase containing a counterion agent and an organic solvent, (c) contacting the polynucleotide with a reversible DNA-binding dye to form a complex between the polynucleotide and the reversible DNA-binding dye, and (d) detecting the complex. Preferred reversible DNA-binding dyes includes DNA intercalating dyes and DNA groove binding dyes. Non-limiting examples of reversible DNA-binding dyes include PICO GREEN, ethidium bromide, propidium iodide, Acrydine orange, 7-aminoactinomycin D, cyanine dyes, Bisbenzimide, Bisbenzimide, Benzoxanthene yellow, Netropsin, SYTO, SYBR Green I, SYBR Green II, SYBR Gold, SYBR DX, OliGreen, CyQuant GR, SYTOX Green, SYTO9, SYTO10, SYTO17, SYBR14, FUN-1, DEAD Red, Hexidium Iodide, Dihydroethidium, Ethidium Homodimer, 9-Amino-6-Chloro-2-Methoxyacridine, DAPI, DIPI, Indole dye, Imidazole dye, Actinomycin D, Hydroxystilbamidine, and LDS 751.

In another aspect, the present invention provides reversible DNA-binding dyes that are used to enhance the detection of polynucleotides. The term “reversible DNA-binding dye” is used herein to include intercalating dyes and DNA groove binding dyes. An “intercalating dye” is defined herein to include a generally planar, aromatic, ring-shaped chromophore molecule which binds to DNA, or other polynucleotide, in a reversible, non-covalent fashion, by insertion between the base pairs of the double helix.

The term “DNA groove binding dye” is defined herein to include those chromophore molecules which reversibly bind by direct interaction with the edges of base pairs in either of the grooves (major or minor) of nucleic acids. These dyes are included in the group comprising non-intercalative DNA binding agents. Non-limiting examples of DNA groove binding dyes include Netropsin (N′-(2-amidinoethyl)-4-(2-guanidinoacetamido)-1,1′-dimethyl-N,4′-bi[pyrrole-2-carboxamide]) (Sigma), Hoechst dye no. 33258 (Bisbenzimide, B-2261, Sigma), Hoechst dye no. 33342, (Bisbenzimide, B2261, Sigma), and Hoechst dye no. 2495 (Benzoxanthene yellow, B-9761, Sigma).

Preferred reversible DNA-binding dyes in the present invention include fluorescent dyes. Non-limiting examples of preferred reversible DNA-binding dyes include PICO GREEN (P-7581, Molecular Probes), ethidium bromide (E-8751, Sigma), propidium iodide (P-4170, Sigma), Acrydine orange (A-6014, Sigma), 7-aminoactinomycin D (A-1310, Molecular Probes), cyanine dyes (e.g., TOTO-1, YOYO-1, BOBO, and POPO-3), SYTO, SYBR Green I, SYBR Green II, SYBR Gold, SYBR DX, OliGreen, CyQuant GR, SYTOX Green, SYTO9, SYTO10, SYTO17, SYBR14, FUN-1, DEAD Red, Hexidium Iodide, Dihydroethidium, Ethidium Homodimer, 9-Amino-6-Chloro-2-Methoxyacridine, DAPI, DIPI, Indole dye, Imidazole dye, palatine chrome black 6BN, Actinomycin D, Hydroxystilbamidine, and LDS 751. Numerous reversible DNA-binding dyes are described in Handbook of Fluorescent Probes and Research Chemicals, Ch. 8.1 (1997) (Molecular Probes, Inc., Eugene, Oreg.); PCT publications WO00166799; WO09919514; WO09810099; W009746714; European Patent Application No. EP 0 634 640 A1; Canadian Patent No. CA 2,119,126; and in the following U.S. Pat. Nos.: 4,716,905; 5,312,921; 5,321,130; 5,410,030; 5,432,134; 5,445,946; 5,646,264; 5,658,735; 5,734,058; 5,760,201; 5,929,227; 6,054,272; 6,162,931; 6,187,787; 6,210,885; and 6,280,933.

In one embodiment, a polynucleotide sample is contacted with a reversible DNA-binding dye, such as a fluorescent intercalating dye, after elution from the separation column. A preferred ratio of dye to DNA is about 1 molecule of dye per 30 base pairs. Preferred dyes (e.g., TOTO) are those that have little or no intrinsic fluorescence and actually exhibit fluorescence only when intercalated into a polynucleotide. The fluorescence can be detected using a conventional fluorescence detector as described herein.

Preferred Intercalating dyes for use in the present invention are those that fluoresce only when bound to dsDNA, ssDNA, or RNA (depending on the dye itself).

“Reversed phase support” refers to a stationary support (including the base material and any chemically bonded phase) for use in liquid chromatography, particularly high performance liquid chromatography (HPLC), which is less polar (e.g., more hydrophobic) than the starting mobile phase.

“Ion-pair (IP) chromatography” refers to a chromatographic method for separating samples in which some or all of the sample components contain functional groups which are ionized or are ionizable. Ion-pair chromatography is typically carried out with a reversed phase column in the presence of an ion-pairing reagent.

“Ion-pairing reagent” is a reagent which interacts with ionized or ionizable groups in a sample to improve resolution in a chromatographic separation. An “ion-pairing agent” refers to both the reagent and aqueous solutions thereof. An ion-pairing agent is typically added to the mobile phase in reversed phase liquid chromatography for optimal separation. The concentration and hydrophobicity of an ion-pairing agent of choice will depend upon the number and types (e.g., cationic or anionic) of charged sites in the sample to be separated.

Ion-Pairing Reversed-Phase Chromatography (IP-RPC) is a powerful form of chromatography used in the separation and analysis of polynucleotides, including DNA (both single and double stranded) and RNA (Eriksson et al., (1986) J. Chromatography 359:265-74). Most reported applications of IP-RPC have been in the context of high performance liquid chromatography (IP-RP-HPLC), but the technology can be accomplished using non-HPLC chromatography systems (U.S. patent application Ser. Nos. 09/318,407 and 09/391,963. Nevertheless, for the sake of simplicity much of the following description will focus on the use of IP-RP-HPLC, a particularly powerful and convenient form of IP-RPC. It is to be understood that this is not intended to limit the scope of the invention, and that generally the methods described can be performed without the use of HPLC, although this will in some cases lead to less than optimal results. IP-RPC is a form of chromatography characterized by the use of a reversed phase (i.e., hydrophobic) stationary phase and a mobile phase that includes an alkylated cation (e.g., triethylammonium) that is believed to form a bridging interaction between the negatively charged polynucleotide and non-polar stationary phase. The alkylated cation-mediated interaction of polynucleotide and stationary phase can be modulated by the polarity of the mobile phase, conveniently adjusted by means of a solvent that is less polar than water, e.g., acetonitrile. In general, a polynucleotide is retained by the separation medium in the presence of counterion agent, and can be eluted by increasing the concentration of a non-polar solvent, Elution can be accomplished in the presence or absence of counterion agent. Performance is enhanced by the use of a non-porous separation medium, as described in U.S. Pat. No. 5,585,236. IP-RP-HPLC (also referred to as MIPC) are described in U.S. Pat. Nos. 5,585,236, 6,066,258 and 6,056,877 and PCT Publication Nos. WO98/48913, WO98/48914, WO/9856797, WO98/56798, incorporated herein by reference in their entirety. MIPC is characterized by the preferred use of solvents and chromatographic surfaces that are substantially free of multivalent cation contamination that can interfere with polynucleotide separation. In the practice of the instant invention, a preferred system for performing IP-RP-HPLC separations is that provided by Transgenomic, Inc. under the trademark WAVE®.

Separation by IP-RP-HPLC occurs at the non-polar surface of a separation medium. In one embodiment, the non-polar surfaces comprise the surfaces of polymeric beads. In an alternative embodiment, the surfaces comprise the surfaces of interstitial spaces in a molded polymeric monolith, described in more detail infra. For purposes of simplifying the description of the invention and not by way of limitation, the separation of polynucleotides using nonporous beads, and the preparation of such beads, will be primarily described herein, it being understood that other separation surfaces, such as the interstitial surfaces of polymeric monoliths, are intended to be included within the scope of this invention.

In general, in order to be suitable for use in IP-RP-HPLC a separation medium should have a surface that is either intrinsically non-polar or bonded with a material that forms a surface having sufficient non-polarity to interact with a counterion agent.

In one aspect of the invention, IP-RP-HPLC detection is accomplished using a column filled with nonporous polymeric beads having an average diameter of about 0.5-100 microns; preferably, 1-10 microns; more preferably, 1-5 microns. Beads having an average diameter of 1.0-3.0 microns are most preferred.

In a preferred embodiment of the invention, the chromatographic separation medium comprises nonporous beads, i.e., beads having a pore size that essentially excludes the polynucleotides being separated from entering the bead, although porous beads can also be used. As used herein, the term “nonporous” is defined to denote a bead that has surface pores having a diameter that is sufficiently small so as to effectively exclude the smallest DNA fragment in the separation in the solvent medium used therein. Included in this definition are polymer beads having these specified maximum size restrictions in their natural state or which have been treated to reduce their pore size to meet the maximum effective pore size required.

The surface conformations of nonporous beads of the present invention can include depressions and shallow pit-like structures that do not interfere with the separation process. A pretreatment of a porous bead to render it nonporous can be effected with any material which will fill the pores in the bead structure and which does not significantly interfere with the IP-RP-HPLC process.

Pores are open structures through which mobile phase and other materials can enter the bead structure. Pores are often interconnected so that fluid entering one pore can exit from another pore. Without intending to be bound by any particular theory, it is believed that pores having dimensions that allow movement of the polynucleotide into the interconnected pore structure and into the bead impair the resolution of separations or result in separations that have very long retention times.

Non-porous polymeric beads useful in the practice of the present invention can be prepared by a two-step process in which small seed beads are initially produced by emulsion polymerization of suitable polymerizable monomers. The emulsion polymerization procedure is a modification of the procedure of Goodwin, et al. (Colloid & Polymer Sci., 252:464-471 (1974)). Monomers which can be used in the emulsion polymerization process to produce the seed beads include styrene, alkyl substituted styrenes, alpha-methyl styrene, and alkyl substituted alpha-methyl styrene. The seed beads are then enlarged and, optionally, modified by substitution with various groups to produce the nonporous polymeric beads of the present invention.

The seed beads produced by emulsion polymerization can be enlarged by any known process for increasing the size of the polymer beads. For example, polymer beads can be enlarged by the activated swelling process disclosed in U.S. Pat. No. 4,563,510. The enlarged or swollen polymer beads are further swollen with a crosslinking polymerizable monomer and a polymerization initiator. Polymerization increases the crosslinking density of the enlarged polymeric bead and reduces the surface porosity of the bead. Suitable crosslinking monomers contain at least two carbon-carbon double bonds capable of polymerization in the presence of an initiator. Preferred crosslinking monomers are divinyl monomers, preferably alkyl and aryl (phenyl, naphthyl, etc.) divinyl monomers and include divinyl benzene, butadiene, etc. Activated swelling of the polymeric seed beads is useful to produce polymer beads having an average diameter ranging-from 1 up to about 100 microns.

Alternatively, the polymer seed beads can be enlarged simply by heating the seed latex resulting from emulsion polymerization. This alternative eliminates the need for activated swelling of the seed beads with an activating solvent. Instead, the seed latex is mixed with the crosslinking monomer and polymerization initiator described above, together with or without a water-miscible solvent for the crosslinking monomer. Suitable solvents include acetone, tetrahydrofuran (THF), methanol, and dioxane. The resulting mixture is heated for about 1-12 hours, preferably about 4-8 hours, at a temperature below the initiation temperature of the polymerization initiator, generally, about 10° C.-80° C., preferably 30° C.-60° C. Optionally, the temperature of the mixture can be increased by 10-20% and the mixture heated for an additional 1 to 4 hours. The ratio of monomer to polymerization initiator is at least 100:1, preferably in the range of about 100:1 to about 500:1, more preferably about 200:1 in order to ensure a degree of polymerization of at least 200. Beads having this degree of polymerization are sufficiently pressure-stable to be used in HPLC applications. This thermal swelling process allows one to increase the size of the bead by about 110-160% to obtain polymer beads having an average diameter up to about 5 microns, preferably about 2-3 microns. The thermal swelling procedure can, therefore, be used to produce smaller particle sizes previously accessible only by the activated swelling procedure.

Following thermal enlargement, excess crosslinking monomer is removed and the particles are polymerized by exposure to ultraviolet light or heat. Polymerization can be conducted, for example, by heating of the enlarged particles to the activation temperature of the polymerization initiator and continuing polymerization until the desired degree of polymerization has been achieved. Continued heating and polymerization allows one to obtain beads having a degree of polymerization greater than 500.

For use in the present invention, packing material disclosed by U.S. Pat. No. 4,563,510 can be modified through substitution of the polymeric beads with alkyl groups or can be used in its unmodified state. For example, the polymer beads can be alkylated with 1 or 2 carbon atoms by contacting the beads with an alkylating agent, such as methyl iodide or ethyl iodide. Alkylation can be achieved by mixing the polymer beads with the alkyl halide in the presence of a Friedel-Crafts catalyst to effect electrophilic aromatic substitution on the aromatic rings at the surface of the polymer blend. Suitable Friedel-Crafts catalysts are well-known in the art and include Lewis acids such as aluminum chloride, boron trifluoride, tin tetrachloride, etc. The beads can be hydrocarbon substituted by substituting the corresponding hydrocarbon halide for methyl iodide in the above procedure, for example.

The term alkyl as used herein in reference to the beads useful in the practice of the present invention is defined to include alkyl and alkyl substituted aryl groups, having from 1 to 1,000,000 carbons, the alkyl groups including straight chained, branch chained, cyclic, saturated, unsaturated nonionic functional groups of various types including aldehyde, ketone, ester, ether, alkyl groups, and the like, and the aryl groups including as monocyclic, bicyclic, and tricyclic aromatic hydrocarbon groups including phenyl, naphthyl, and the like. Methods for alkyl substitution are conventional and well-known in the art and are not an aspect of this invention. The substitution can also contain hydroxy, cyano, nitro groups, or the like which are considered to be non-polar, reverse phase functional groups.

Non-limiting examples of base polymers suitable for use in producing such polymer beads include mono- and di-vinyl substituted aromatics such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(styrene-divinylbenzene) and poly(ethylvinylbenzene-divinylbenzene). Methods for making beads from these polymers are conventional and well known in the art (for example, see U.S. Pat. No. 4,906,378). The physical properties of the surface and near-surface areas of the beads are the primary determinant of chromatographic efficiency. The polymer, whether derivatized or not, should provide a nonporous, non-reactive, and non-polar surface for the IP-RP-HPLC separation. In a particularly preferred embodiment of the invention, the separation medium consists of octadecyl modified, nonporous alkylated poly(styrene-divinylbenzene) beads. Separation columns employing these particularly preferred beads, referred to as DNASep® columns, are commercially available from Transgenomic, Inc.

A separation bead used in the invention can comprise a nonporous particle which has non-polar molecules or a non-polar polymer attached to or coated on its surface. In general, such beads comprise nonporous particles which have been coated with a polymer or which have substantially all surface substrate groups reacted with a non-polar hydrocarbon or substituted hydrocarbon group, and any remaining surface substrate groups endcapped with a tri(lower alkyl)chlorosilane or tetra(lower alkyl)dichlorodisilazane as described in U.S. Pat. No. 6,056,877.

The nonporous particle is preferably an inorganic particle, but can be a nonporous organic particle. The nonporous particle can be, for example, silica, silica carbide, silica nitrite, titanium oxide, aluminum oxide, zirconium oxide, carbon, insoluble polysaccharides such as cellulose, or diatomaceous earth, or any of these materials which have been modified to be nonporous. Examples of carbon particles include diamond and graphite which have been treated to remove any interfering contaminants. The preferred particles are essentially non-deformable and can withstand high pressures. The nonporous particle is prepared by known procedures. The preferred particle size is about 0.5-100 microns; preferably, 1-10 microns; more preferably, 1-5 microns. Beads having an average diameter of 1.0-3.0 microns are most preferred.

Because the chemistry of preparing conventional silica-based reverse phase HPLC materials is well-known, most of the description of non-porous beads suitable for use in the instant invention is presented in reference to silica. It is to be understood, however, that other nonporous particles, such as those listed above, can be modified in the same manner and substituted for silica. For a description of the general chemistry of silica, see Poole, Colin F. and Salwa K. Poole, Chromatography Today, Elsevier:N.Y. (1991), pp. 313-342 and Snyder, R. L. and J. J. Kirkland, Introduction to Modern Liquid Chromatography, 2^{nd} ed., John Wiley & Sons, Inc.: New York (1979), pp. 272-278, the disclosures of which are hereby incorporated herein by reference in their entireties.

The nonporous beads of the invention are characterized by having minimum exposed silanol groups after reaction with the coating or silating reagents. Minimum silanol groups are needed to reduce the interaction of the DNA with the substrate and also to improve the stability of the material in a high pH and aqueous environment. Silanol groups can be harmful because they can repel the negative charge of the DNA molecule, preventing or limiting the interaction of the DNA with the stationary phase of the column. Another possible mechanism of interaction is that the silanol can act as ion exchange sites, taking up metals such as iron (III) or chromium (III). Iron (III) or other metals which are trapped on the column can distort the DNA peaks or even prevent DNA from being eluted from the column.

Silanol groups can be hydrolyzed by the aqueous-based mobile phase. Hydrolysis will increase the polarity and reactivity of the stationary phase by exposing more silanol sites, or by exposing metals that can be present in the silica core. Hydrolysis will be more prevalent with increased underivatized silanol groups. The effect of silanol groups on the DNA separation depends on which mechanism of interference is most prevalent. For example, iron (III) can become attached to the exposed silanol sites, depending on whether the iron (III) is present in the eluent, instrument or sample.

The effect of metals can only occur if metals are already present within the system or reagents. Metals present within the system or reagents can get trapped by ion exchange sites on the silica. However, if no metals are present within the system or reagents, then the silanol groups themselves can cause interference with DNA separations. Hydrolysis of the exposed silanol sites by the aqueous environment can expose metals that might be present in the silica core.

Fully hydrolyzed silica contains a concentration of about 8 μmoles of silanol groups per square meter of surface. At best, because of steric considerations, a maximum of about 4.5 μmoles of silanol groups per square meter can be reacted, the remainder of the silanol being sterically shielded by the reacted groups. Minimum silanol groups is defined as reaching the theoretical limit of or having sufficient shield to prevent silanol groups from interfering with the separation.

Numerous methods exist for forming nonporous silica core particles. For example, sodium silicate solution poured into methanol will produce a suspension of finely divided spherical particles of sodium silicate. These particles are neutralized by reaction with acid. In this way, globular particles of silica gel are obtained having a diameter of about 1-2 microns. Silica can be precipitated from organic liquids or from a vapor. At high temperature (about 2000° C.), silica is vaporized, and the vapors can be condensed to form finely divided silica either by a reduction in temperature or by using an oxidizing gas. The synthesis and properties of silica are described by R. K. Iler in The Chemistry of Silica, Solubility, Polymerization, Colloid and Surface Properties, and Biochemistry, John Wiley & Sons: New York (1979).

W. Stöber et al. described controlled growth of monodisperse silica spheres in the micron size range in J. Colloid and Interface Sci., 26:62-69 (1968). Stöber et al. describe a system of chemical reactions which permit the controlled growth of spherical silica particles of uniform size by means of hydrolysis of alkyl silicates and subsequent condensation of silicic acid in alcoholic solutions. Ammonia is used as a morphological catalyst. Particle sizes obtained in suspension range from less than 0.05 μm to 2 μm in diameter.

To prepare a nonporous bead, the nonporous particle can be coated with a polymer or reacted and endcapped so that substantially all surface substrate groups of the nonporous particle are blocked with a non-polar hydrocarbon or substituted hydrocarbon group. This can be accomplished by any of several methods described in U.S. Pat. No. 6,056,877. Care should be taken during the preparation of the beads to ensure that the surface of the beads has minimum silanol or metal oxide exposure and that the surface remains nonporous. Nonporous silica core beads can be obtained from Micra Scientific (Northbrook, Ill.) and from Chemie Uetikkon (Lausanne, Switzerland).

Another example of a suitable stationary support is a wide pore silica-based alkylated support as described in U.S. Pat. No. 6,379,889.

In another embodiment of the present invention, the IP-RP-HPLC separation medium can be in the form of a polymeric monolith, e.g., a rod-like monolithic column. A monolith is a polymer separation media, formed inside a column, having a unitary structure with through pores or interstitial spaces that allow eluting solvent and analyte to pass through and which provide the non-polar separation surface, as described in U.S. Pat. No. 6,066,258 and U.S. patent application Ser. No. 09/562,069. Monolithic columns, including capillary columns, can also be used, such as disclosed in U.S. Pat. No. 6,238,565; U.S. patent application Ser. No. 09/562,069 filed May 1, 2000; the PCT application WO00/15778; and by Huber et al (Anal. Chem. 71:3730-3739 (1999)). The interstitial separation surfaces can be porous, but are preferably nonporous. The separation principles involved parallel those encountered with bead-packed columns. As with beads, pores traversing the monolith must be compatible with and permeable to DNA. In a preferred embodiment, the rod is substantially free of contamination capable of reacting with DNA and interfering with its separation, e.g., multivalent cations.

A molded polymeric monolith rod that can be used in practicing the present invention can be prepared, for example, by bulk free radical polymerization within the confines of a chromatographic column. The base polymer of the rod can be produced from a variety of polymerizable monomers. For example, the monolithic rod can be made from polymers, including mono- and di-vinyl substituted aromatic compounds such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(glycidyl methacrylate-co-ethylene dimethacrylate), poly(styrene-divinylbenzene) and poly(ethylvinylbenzene-divinylbenzene. The rod can be unsubstituted or substituted with a substituent such as a hydrocarbon alkyl or an aryl group. The alkyl group optionally has 1 to 1,000,000 carbons inclusive in a straight or branched chain, and includes straight chained, branch chained, cyclic, saturated, unsaturated nonionic functional groups of various types including aldehyde, ketone, ester, ether, alkyl groups, and the like, and the aryl groups includes as monocyclic, bicyclic, and tricyclic aromatic hydrocarbon groups including phenyl, naphthyl, and the like. In a preferred embodiment, the alkyl group has 1-24 carbons. In a more preferred embodiment, the alkyl group has 1-8 carbons. The substitution can also contain hydroxy, cyano, nitro groups, or the like which are considered to be non-polar, reverse phase functional groups. Methods for hydrocarbon substitution are conventional and well-known in the art and are not an aspect of this invention. The preparation of polymeric monoliths is by conventional methods well known in the art as described in the following references: Wang et al.(1994) J. Chromatog. A 699:230; Petro et al. (1-996) Anal. Chem. 68:315 and U.S. Pat. Nos. 5,334,310; 5,453,185 and 5,522,994. Monolith or rod columns are commercially available form Merck & Co (Darmstadt, Germany).

The separation medium can take the form of a continuous monolithic silica gel. A molded monolith can be prepared by polymerization within the confines of a chromatographic column (e.g., to form a rod) or other containment system. A monolith is preferably obtained by the hydrolysis and polycondensation of alkoxysilanes. A preferred monolith is derivatized in order to produce non-polar interstitial surfaces. Chemical modification of silica monoliths with ocatdecyl, methyl or other ligands can be carried out. An example of a preferred derivatized monolith is one which is polyfunctionally derivatized with octadecylsilyl groups. The preparation of derivatized silica monoliths can be accomplished using conventional methods well known in the art as described in the following references which are hereby incorporated in their entirety herein: U.S. Pat. No. 6,056,877, Nakanishi, et al., J. Sol-Gel Sci Technol. 8:547 (1997); Nakanishi, et al., Bull, Chem Soc. Jpn. 67:1327 (1994); Cabrera, et al., Trends Analytical Chem. 17:50 (1998); Jinno, et al., Chromatographia 27:288 (1989).

MIPC is characterized by the preferred use of a separation medium that is substantially free of metal contaminants or other contaminants that can bind DNA. Preferred beads and monoliths have been produced under conditions where precautions have been taken to substantially eliminate any multivalent cation contaminants (e.g. Fe(III), Cr(III), or colloidal metal contaminants), including a decontamination treatment, e.g., an acid wash treatment. Only very pure, non-metal containing materials should be used in the production of the beads in order to minimize the metal content of the resulting beads.

In addition to the separation medium being substantially metal-free, to achieve optimum peak separation the separation column and all process solutions held within the column or flowing through the column are preferably substantially free of multivalent cation contaminants (e.g. Fe(III), Cr(III), and colloidal metal contaminants). As described in U.S. Pat. Nos. 5,772,889, 5,997,742 and 6,017,457, this can be achieved by supplying and feeding solutions that enter the separation column with components that have process solution-contacting surfaces made of material which does not release multivalent cations into the process solutions held within or flowing through the column, in order to protect the column from multivalent cation contamination. The process solution-contacting surfaces of the system components are preferably material selected from the group consisting of titanium, coated stainless steel, passivated stainless steel, and organic polymer. Metals found in stainless steel, for example, do not harm the separation, unless they are in an oxidized or colloidal partially oxidized state. For example, 316 stainless steel frits are acceptable in column hardware, but surface oxidized stainless steel frits harm the DNA separation.

For additional protection, multivalent cations in mobile phase solutions and sample solutions entering the column can be removed by contacting these solutions with multivalent cation capture resin before the solutions enter the column to protect the separation medium from multivalent cation contamination. The multivalent capture resin is preferably cation exchange resin and/or chelating resin.

Trace levels of multivalent cations anywhere in the solvent flow path can cause a significant deterioration in the resolution of the separation after multiple uses of an IP-RP-HPLC column. This can result in increased cost caused by the need to purchase replacement columns and increased downtime. Therefore, effective measures are preferably taken to prevent multivalent metal cation contamination of the separation system components, including separation media and mobile phase contacting. These measures include, but are not limited to, washing protocols to remove traces of multivalent cations from the separation media and installation of guard cartridges containing cation capture resins, in line between the mobile phase reservoir and the IP-RP-HPLC column. These, and similar measures, taken to prevent system contamination with multivalent cations have resulted in extended column life and reduced analysis downtime.

There are two places where multivalent-cation-binding agents, e.g., chelators, are used in MIPC separations. In one embodiment, these binding agents can be incorporated into a solid through which the mobile phase passes. Contaminants are trapped before they reach places within the system that can harm the separation. In these cases, the functional group is attached to a solid matrix or resin (e.g., a flow-through cartridge, usually an organic polymer, but sometimes silica or other material). The capacity of the matrix is preferably about 2 mequiv./g. An example of a suitable chelating resin is available under the trademark CHELEX 100 (Dow Chemical Co.) containing an iminodiacetate functional group.

In another embodiment, the multivalent cation-binding agent can be added to the mobile phase. The binding functional group is incorporated into an organic chemical structure. The preferred multivalent cation-binding agent fulfills three requirements. First, it is soluble in the mobile phase. Second, the complex with the metal is soluble in the mobile phase. Multivalent cation-binding agents such as EDTA fulfill this requirement because both the chelator and the multivalent cation-binding agent-metal complex contain charges, which makes them both water-soluble. Also, neither precipitate when acetonitrile, for example, is added. The solubility in aqueous mobile phase can be enhanced by attaching covalently bound ionic functionality, such as, sulfate, carboxylate, or hydroxy. A preferred multivalent cation-binding agent can be easily removed from the column by washing with water, organic solvent or mobile phase. Third, the binding agent must not interfere with the chromatographic process.

The multivalent cation-binding agent can be a coordination compound. Examples of preferred coordination compounds include water soluble chelating agents and crown ethers. Non-limiting examples of multivalent cation-binding agents which can be used in the present invention include acetylacetone, alizarin, aluminon, chloranilic acid, kojic acid, morin, rhodizonic acid, thionalide, thiourea, α-furildioxime, nioxime, salicylaldoxime, dimethylglyoxime, a-furildioxime, cupferron, α-nitroso-β-naphthol, nitroso-R-salt, diphenylthiocarbazone, diphenylcarbazone, eriochrome black T, PAN, SPADNS, glyoxal-bis(2-hydroxyanil), murexide, α-benzoinoxime, mandelic acid, anthranilic acid, ethylenediamine, glycine, triaminotriethylamine, thionalide, triethylenetetramine, EDTA, metalphthalein, arsonic acids, α,α′-bipyridine, 4-hydroxybenzothiazole, 8-hydroxyquinaldine, 8-hydroxyquinoline, 1,10-phenanthroline, picolinic acid, quinaldic acid, α,α′,α″-terpyridyl, 9-methyl-2,3,7-trihydroxy-6-fluorone, pyrocatechol, salicylic acid, tiron, 4-chloro-1,2-dimercaptobenzene, dithiol, mercaptobenzothiazole, rubeanic acid, oxalic acid, sodium diethyldithiocarbarbamate, and zinc dibenzyldithiocarbamate. These and other examples are described by Perrin in Organic Complexing Reagents: Structure, Behavior, and Application to Inorganic Analysis, Robert E. Krieger Publishing Co. (1964). In the present invention, a preferred multivalent cation-binding agent is EDTA.

To achieve high-resolution chromatographic separations of polynucleotides, it is generally necessary to tightly pack the chromatographic column with the solid phase polymer beads. Any known method of packing the column with a column packing material can be used in the present invention to obtain adequate high-resolution separations. Typically, a slurry of the polymer beads is prepared using a solvent having a density equal to or less than the density of the polymer beads. The column is then filled with the polymer bead slurry and vibrated or agitated to improve the packing density of the polymer beads in the column. Mechanical vibration or sonication is typically used to improve packing density.

For example, to pack a 50×4.6 mm I.D. column, 2.0 grams of beads can be suspended in 10 mL of methanol with the aid of sonication. The suspension is then packed into the column using 50 mL of methanol at 8,000 psi of pressure. This improves the density of the packed bed.

There are several types of counterions suitable for use with IP-RP-HPLC. These include a mono-, di-, or trialkylamine that can be protonated to form a positive counter charge or a quaternary alkyl substituted amine that already contains a positive counter charge. The alkyl substitutions may be uniform (for example, triethylammonium acetate or tetrapropylammonium acetate) or mixed (for example, propyldiethylammonium acetate). The size of the alkyl group may be small (methyl) or large (up to 30 carbons) especially if only one of the substituted alkyl groups is large and the others are small. For example octyldimethylammonium acetate is a suitable counterion agent. Preferred counterion agents are those containing alkyl groups from the ethyl, propyl or butyl size range.

Without intending to be bound by any particular theory, it is believed the alkyl group functions by imparting a nonpolar character to the DNA through an ion pairing process so that the DNA can interact with the nonpolar surface of the separation media. The requirements for the degree of nonpolarity of the counterion-DNA pair depends on the polarity of the separation media, the solvent conditions required for separation, the particular size and type of fragment being separated. For example, if the polarity of the separation media is increased, then the polarity of the counterion agent may have to be adjusted to match the polarity of the surface and increase interaction of the counterion-DNA pair. In general, as the size and hydrophobicity of the alkyl group is increased, the separation is less influenced by DNA sequence and base composition, but rather is based predominately on DNA sequence length.

In some cases, it may be desired to increase the range of concentration of organic solvent used to perform the separation. For example, increasing the alkyl chain length on the counterion agent will increase the nonpolarity of the counterion-DNA pair resulting in the need to either increase the concentration of the mobile phase organic solvent, or increase the strength of the organic solvent type, e.g., acetonitrile is about two times more effective than methanol for eluting DNA. There is a positive correlation between concentration of the organic solvent required to elute a fragment from the column and the length of the fragment. However, at high organic solvent concentrations, the polynucleotide can precipitate. To avoid precipitation, a more non-polar organic solvent and/or a smaller counterion alkyl group can be used. The alkyl group on the counterion agent can also be substituted with halides, nitro groups, or the like to modulate polarity.

The mobile phase preferably contains a counterion agent. Typical counterion agents include trialkylammonium salts of organic or inorganic acids, such as lower alkyl primary, secondary, and lower tertiary amines, lower trialkyammonium salts and lower quaternary alkyalmmonium salts. Lower alkyl refers to an alkyl radical of one to six carbon atoms, as exemplified by methyl, ethyl, n-butyl, i-butyl, t-butyl, isoamyl, n-pentyl, and isopentyl. Examples of counterion agents include octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyldiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, and tetrabutylammonium acetate. Although the anion in the above examples is acetate, other anions may also be used, including carbonate, phosphate, sulfate, nitrate, propionate, formate, chloride, and bromide, or any combination of cation and anion. These and other agents are described by Gjerde, et al. in Ion Chromatography, 2nd Ed., Dr. Alfred Hüthig Verlag Heidelberg (1987). In a particularly preferred embodiment of the invention the counterion is tetrabutylammonium bromide (TBAB) is preferred, although other quaternary ammonium reagents such as tetrapropyl or tetrabutyl ammonium salts can be used. Alternatively, a trialkylammonium salt, e.g., triethylammonium acetate (TEAA) can be used. The pH of the mobile phase is preferably within the range of about pH 5 to about pH 9, and optimally within the range of about pH 6 to about pH 7.5.

Depending on the conditions, IP-RP-HPLC separates double stranded polynucleotides by size or by base pair sequence and is therefore a preferred separation technology for detecting the presence of particular fragments of DNA of interest. The chromatographic profile can be in the form of a visual display, a printed representation of the data or the original data stream.

The IP-RP-HPLC retention times of double stranded DNA fragments can be predicted using software such as Wavemaker™ software (Transgenomic) or Star workstation software (Varian). These programs allow prediction of the retention time based on the length of a DNA fragment for a given set of elution conditions (U.S. Pat. Nos. 6,287,822 and 6,197,516; and in U.S. patent application Ser. No. 09/469,551 filed Dec. 22, 1999; and PCT publications WO99/07899 and WO 01/46687).

As the use and understanding of IP-RP-HPLC developed it became apparent that when IP-RP-HPLC analyses were carried out at a partially denaturing temperature, i.e., a temperature sufficient to denature a heteroduplex at the site of base pair mismatch, homoduplexes could be separated from heteroduplexes having the same base pair length (Hayward-Lester, et al., Genome Research 5:494 (1995); Underhill, et al., Proc. Natl. Acad. Sci. U.S.A 93:193 (1996); Doris, et al., DHPLC Workshop, Stanford University, (1997)). Thus, the use of denaturing high performance liquid chromatography (DHPLC) was applied to mutation detection (Underhill, et al., Genome Research 7:996 (1997); Liu, et al., Nucleic Acid Res., 26;1396 (1998)).

When mixtures of DNA fragments are mixed with an ion pairing agent and applied to a reverse phase separation column, they are separated by size, the smaller fragments eluting from the column first. IP-RP-HPLC, when performed at a temperature which is sufficient to partially denature a heteroduplex, is referred to as DHPLC. DHPLC is also referred to in the art as “Denaturing Matched Ion Polynucleotide Chromatography” (DMIPC).

DHPLC for separating heteroduplex (double-stranded nucleic acid molecules having less than 100% sequence complementarity) and homoduplex (double-stranded nucleic acid molecules having 100% sequence complementarity) nucleic acid samples (e.g., DNA or RNA) in a mixture is described in U.S. Pat. Nos. 5,795,976; 6,287,822; and 6,379,889. In the separation method, a mixture containing both heteroduplex and homoduplex nucleic acid samples is applied to a stationary reversed phase support. The sample mixture is then eluted with a mobile phase containing an ion-pairing reagent and an organic solvent. Sample elution is carried out under conditions effective to at least partially denature the duplexes and results in the separation of the heteroduplex and homoduplex molecules.

The term “hybridization” refers to a process of heating and cooling a double stranded DNA (dsDNA) sample, e.g., heating to 95° C. followed by slow cooling. The heating process causes the DNA strands to denature. Upon cooling, the strands re-combine, or re-anneal, into duplexes.

In preparing a set of DNA fragments for analysis by DHPLC, it is usually assumed that all of the fragments have the same length since they are typically generated using the same set of PCR primers. It is further usually assumed that the fragments are eluted under essentially the same conditions of temperature and solvent gradient. The pattern or shape of the chromatographic separation profile consists of peaks representing the detector response as various species elution during the separation process. The profile is determined by, for example, the number, height, width, symmetry and retention time of peaks. Other patterns can be observed, such as 3 or 2 peaks. The profile can also include poorly resolved shoulders. The shape of the profile contains useful information about the nature of the sample. The pattern or shape of the resulting chromatogram will be influenced by the type and location of the mutation. Each mutation (e.g. single nucleotide polymorphism (SNP)) has a corresponding elution profile, or signature, at a given set of elution conditions of temperature and gradient.

In IP-RP-HPLC and DHPLC, the length and diameter of the separation column, as well as the system mobile phase pressure and temperature, and other parameters, can be varied. An increase in the column diameter was found to increase resolution of polynucleotide fragments in IP-RP-HPLC and DHPLC (U.S. Pat. No. 6,372,142; WO 01/19485). Size-based separation of DNA fragments can also be performed using batch methods and devices as disclosed in U.S. Pat. Nos. 6,265,168; 5,972,222; and 5,986,085.

In DHPLC, the mobile phase typically contains an ion-pairing agent (i.e. a counter ion agent) and an organic solvent. Ion-pairing agents for use in the method include lower primary, secondary and tertiary amines, lower trialkylammonium salts such as triethylammonium acetate and lower quaternary ammonium salts. Typically, the ion-pairing reagent is present at a concentration between about 0.05 and 1.0 molar. Organic solvents for use in the method include solvents such as methanol, ethanol, 2-propanol, acetonitrile, and ethyl acetate.

In one embodiment of DHPLC, the mobile phase for carrying out the separation contains less than about 40% by volume of an organic solvent and greater than about 60% by volume of an aqueous solution of the ion-pairing agent. In a preferred embodiment, elution is carried out using a binary gradient system.

Partial denaturation of heteroduplex molecules can be carried out in a variety of ways such as alteration of pH or salt concentration, use of denaturing agents, or elevation in temperature. Temperatures for carrying out the separation are typically between about 500 and 70° C. and preferably between about 550 and 65° C. The preferred temperature is sequence dependent. In carrying out a separation of GC-rich heteroduplex and homoduplex molecules, for example, a higher temperature is preferred.

A variety of liquid chromatography systems are available that can be used for conducting DHPLC. These systems typically include software for operating the chromatography components, such as pumps, heaters, mixers, fraction collection devices, injector. Examples of software for operating a chromatography apparatus include HSM Control System (Hitachi), ChemStation (Agilent), VP data system (Shimadzu), Millennium32 Software (Waters), Duo-Flow software (Bio-Rad), and Star workstation (Varian). Examples of preferred liquid chromatography systems for carrying out DHPLC include the WAVE® DNA Fragment Analysis System (Transgenomic) and the Varian ProStar Helix™ System (Varian).

In carrying out DHPLC analysis, the operating temperature and the mobile phase composition can be determined by trial and error. However, these parameters are preferably obtained using software. Computer software that can be used in carrying out DHPLC is disclosed in the following patents and patent applications: U.S. Pat. Nos. 6,287,822; 6,197,516; U.S. patent application Ser. No. 09/469,551 filed Dec. 22, 1999; and in WO0146687 and WO0015778. Examples of software for predicting the optimal temperature for DHPLC analysis are disclosed by Jones et al. in Clinical Chem. 45:113-1140 (1999) and in the website having the address of http://insertion.stanford.edu/melt.html. Examples of a commercially available software include WAVEMaker® software and Navigator™ software (Transgenomic).

Suitable separation media for performing DHPLC are described in the following U.S. patents and patent applications: U.S. Pat. Nos. 6,379,889; 6,056,877; 6,066,258; 5,453,185; 5,334,310; U.S. patent application Ser. No. 09/493,734 filed Jan. 28, 2000; U.S. patent application Ser. No. 09/562,069 filed May 1, 2000; and in the following PCT applications: WO98/48914; WO98/48913; PCT/US98/08388; PCT/US00/11795. Examples of suitable media include separation beads and monolithic rods. An example of a suitable column based on a polymeric stationary support is the DNASep® column (Transgenomic). Examples of suitable columns based on a silica stationary support include the Microsorb Analytical column (Varian and Rainin) and “ECLIPSE dsDNA” (Hewlett Packard, Newport, Del.).

A “Mutation standard” is defined herein to include a mixture of DNA species that when hybridized and analyzed by DHPLC, produce previously characterized mutation separation profiles which can be used to evaluate the performance of the chromatography system. Mutation standards can be obtained commercially (e.g. a WAVE® System Low Range Mutation Standard, part no. 700210, GCH338 Mutation Standard (part no. 700215), and HTMS219 Mutation Standard (part no. 700220) are available from Transgenomic. A 209 bp mutation standard is also available from Varian, Inc. The 209 base pair mutation standard comprises a 209-bp fragment from the human Y chromosome locus DYS217 (GenBank accession number S76940)).

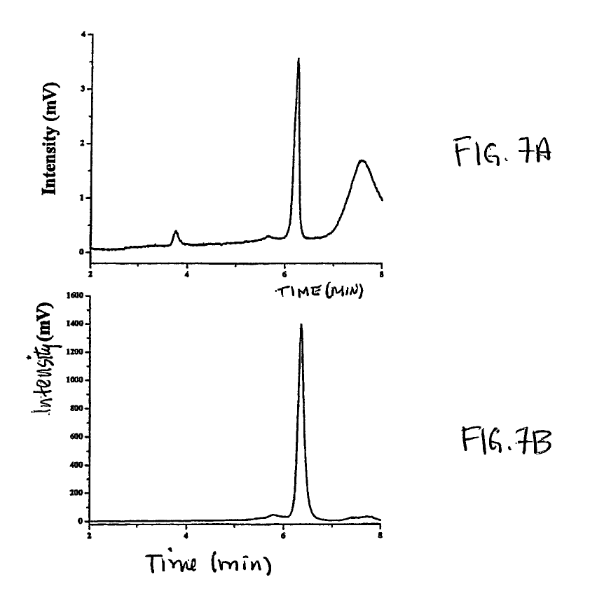

Analysis of a 209 bp Mutation Standard (part no. 700210, Transgenomic) is illustrated schematically in FIG. 7. Prior to injection of the mixture onto the separation column, the mutation standard is preferably hybridized as shown in the scheme 300. The hybridization process created two homoduplexes and two heteroduplexes. As shown in the mutation separation profile 302, the hybridization product was separated using DHPLC. The two lower retention time peaks represent the two heteroduplexes and the two higher retention time peaks represent the two homoduplexes. The two homoduplexes separate because the A-T base pair denatures at a lower temperature than the C-G base pair. Without wishing to be bound by theory, the results are consistent with a greater degree of denaturation in one duplex and/or a difference in the polarity of one partially denatured heteroduplex compared to the other, resulting in a difference in retention time on the reverse-phase separation column.

In another aspect, the invention concerns kits for detecting polynucleotides. A kit of the invention can include one or more of the following:

in a separate container, intercalating dye reagent as described herein. The dye reagent is preferably a nucleic acid stain. Examples of suitable dye reagent include SYBR Green 1, SYBR Green II, SYBR Gold, and mixtures thereof;

in a separate container, a buffer solution for diluting an intercalating dye reagent;

a reactor for mixing intercalating dye reagent with mobile phase eluting from a reverse phase liquid chromatography column;

a reverse-phase liquid chromatography column;

a pump for use with a post-column reactor;

a detector for detecting intercalating dye bound to polynucleotide, for example, a fluorescence detector;

conduit for connecting a post-column reactor to a separation column;

in a separate container, a standard mixture of polynucleotides. Examples include single stranded, double stranded polynucleotides. The polynucleotides can be DNA or RNA.

Another example of a standard mixture is a mutation standard;

Instructional material concerning the use of a post-column reactor and intercalating dyes in a liquid chromatographic system.

It will be appreciated that the inventive concepts herein can be applied to other separation methods, such as conventional capillary electrophoresis. The matrices, electrical field and other conditions for capillary electrophoresis of polynucleotides are well known (such as described in U.S. Pat. Nos. 5,073,239; 5,874,213). U.S. Pat. No. 5,633,129 describes the separation of heteroduplex and homoduplex DNA for mutation detection using constant denaturant capillary electrophoresis. In a further aspect of the present invention, intercalating dyes, as described herein, are contacted with polynucleotides after separation by capillary electrophoresis, and detected (e.g. using fluorescent detection). A preferred capillary electrophoresis system incorporates a modification (such as described in U.S. Pat. No. 5,310,463) in which there is an electrophoretic separation capillary containing a fluid defining a bore therein through which a sample travels and separates into components. The tube has a side wall defining a through hole therein which is surrounded by a medium including an intercalating dye. The dye is introduced into the capillary through the hole by means of gravity, pressure or electroosmosis. In a preferred embodiment of the present invention, one or more intercalating dyes, as described herein, are contacted with polynucleotides after separation by capillary electrophoresis, and detected using conventional fluorescent detection.

In further applications of the method of the invention, mass spectral analysis can be performed downstream of a post-column reactor as described herein. Applicants have observed that the intercalated dye does not affect mass spectral analysis.

The elucidation of the mechanisms involved in tumor formation and growth necessitates the investigation of multiple genes in the human genome. Detection of somatic mutations responsible for tumor progression requires a methodology capable of distinguishing a few mutant gene products in the presence of a vast majority of wild type products. Sequencing is commonly used to determine the nucleotide composition of an allele, however this approach cannot be used to detect low levels of mutations in an exceeding large population of wild type alleles. The technique of DHPLC offers a rapid, inexpensive, and accurate means of monitoring and detecting genetic variations. DHPLC analysis with UV detection can routinely detect a sequence variation at a level of about 5%. By use of the present invention, Applicants have been able to detect a sequence variation as low as 0.1%. This increase in sensitivity by two orders of magnitude over that of UV analysis does not require labeled primers and therefore can be used with any PCR. product. The present invention can therefore be used for the discovery and monitoring of nucleotide variants in genes involved in the pathway of cancer progression.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All patent applications, patents, and literature references cited in this specification are hereby incorporated by reference in their entirety. In case of conflict or inconsistency, the present description, including definitions, will control. Unless mentioned otherwise, the techniques employed or contemplated herein are standard methodologies well known to one of ordinary skill in the art. The materials, methods and examples are illustrative only and not limiting. All numerical ranges in this specification are intended to be inclusive of their upper and lower limits. Other features of the invention will become apparent in the course of the following descriptions of exemplary embodiments which are given for illustration of the invention and are not intended to be limiting thereof. Procedures described in the past tense in the Examples below have been carried out in the laboratory. Procedures described in the present tense have not yet been carried out in the laboratory, and are constructively reduced to practice with the filing of this application.

### Example 1

### Detection of Double-stranded Polynucleotides

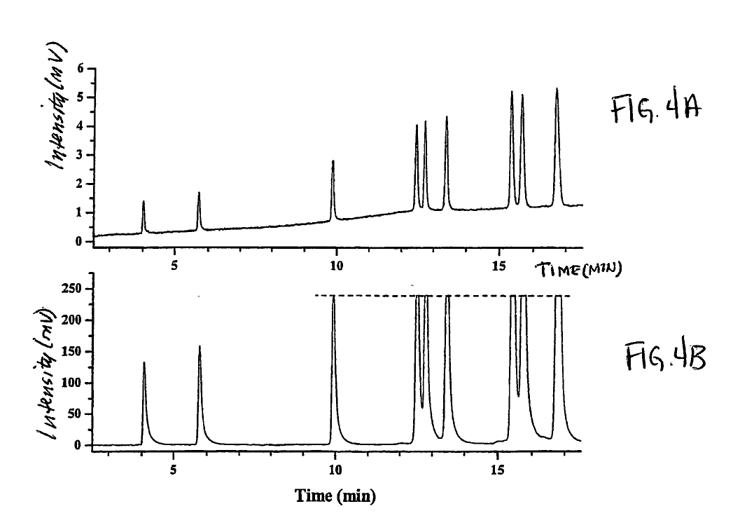

In this example, the sample consisted of a pUC18 HAEIII digest Sizing Standard (0.0485 μg DNA/μl) (part no. 560078, Transgenomic). The nine fragments eluted in order of size (in base pairs) 80, 102, 174, 257, 267, 298, 434, 458, 587, as shown by the nine peaks with retention times ranging from about 4.1 min to about 16.8 min, respectively (FIG. 4). IP-RP-HPLC was performed using a WAVE®) Model 2100A chromatography system (Transgenomic) equipped with a DNASep® cartridge (4.6 mm ID×50 mm) (Transgenomic). The injection volume was 5 μl (0.242 μg DNA). The mobile phase consisted of Buffer A: 0.1 M TEM (Transgenomic) and Buffer B: 0.1 M TEM in 25% acetonitrile, pH 7 (Transgenomic).

The intercalating dye solution consisted of 100 μl SYBR Gold dye (S-11494, Molecular Probes, Eugene, Oreg.) dissolved in 1 liter of buffer (10 mM Tris-HCl (part no. 15568-025, Invitrogen, Carlsbad, Calif.), 1 mM EDTA (ED, Sigma-Aldrich, St Louis, Mo.), pH=8.0 (adjusted with NaOH (S-5881, Sigma)). (The manufacturer indicated that the dye stock solution is 1000X. The concentration in the working dye solution was therefore 10X.)

The elution gradient was as follows:

| Time | % A | % B |
|---|---|---|
| 0.0 | 64.0 | 36.0 |
| 0.5 | 59.0 | 41.0 |
| 18.5 | 30.0 | 70.0 |
| 18.6 | 0.0 | 100.0 |
| 19.1 | 0.0 | 100.0 |
| 19.2 | 64.0 | 36.0 |
| 21.5 | 64.0 | 36.0 |

The run time was 23.0 min. at a flow rate of 0.7 ml/min. The column temperature was 50.0° C.

Detector 1 (Channel 1) was a Hitachi L-7400 set at 260 nm (chromatogram shown in FIG. 4A). Detector 2 (Channel 2), positioned downstream of the post-column reactor, was a Hitachi L7485 fluorescence detector, with an excitation wavelength of 497 nm and an emission wavelength of 535 nm (chromatogram shown in FIG. 4B).

The post-column reactor included the following components: an SSI series 1 pump with a back pressure of about 1000 psi and with an intercalating dye solution flow rate of 0.1 ml/min; a “tee” junction (Upchurch Scientific model number P-713); two check valves, inline ¼-28 fitting style, inlet (Upchurch Scientific model number P-3401); a check valve, inline ¼-28 fitting style, outlet (Upchurch Scientific model number P-3402); assorted tubing and fittings. The “tee” was located after the absorbance detector. The tubing from the SSI pump to the mixing tee included a coil of approximately 3 ft of 0.50 mm ID of PEEK tubing. A 3 ft length of capillary tubing (75 μm ID) was inserted into this tubing. The outer PEEK tubing allowed attachment to the various components of the mixing tee with regular HPLC fittings, and acted as a support for the capillary tubing.

The detector signal for fluorescence was off scale for the fragments above 174 bp (FIG. 4B).

### Example 2

### Detection of Diluted Mixture of DNA Sizing Standards

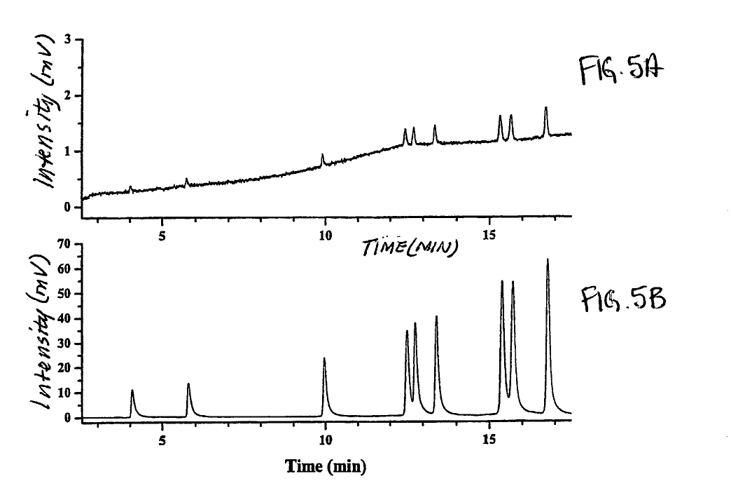

The sample was a 1:10 dilution of the Sizing Standard from Example 1. The buffer used for dilution was 40 mM Tris-HCl, pH=8.0. The injection volume was 5 μl (0.0242 μg DNA). The column was eluted using the same conditions as described in Example 1. The sample was monitored using UV detection (FIG. 5A) and fluorescence detection (FIG. 5B).

The signal enhancement of the post-column intercalation system over the absorbance detector is clearly evident. The noise associated with the fluorescence was 0.00858mv/channel as compared to 0.0133mv/channel for UV absorbance detection. As an example, the signal-to-noise ratio for the 587 peak was about 41.1 for UV detection as compared to about 7240 for fluorescence detection.

### Example 3

### Detection of Diluted DNA Sizing Standards

A 1:80 dilution of the DNA Sizing Standard from Example 1 was prepared. The buffer used for dilution was 40 mM Tris-HCl, pH=8.0. A sample (injection volume 5 μl (0.00303 μg DNA)) was analyzed as described in Example 1.

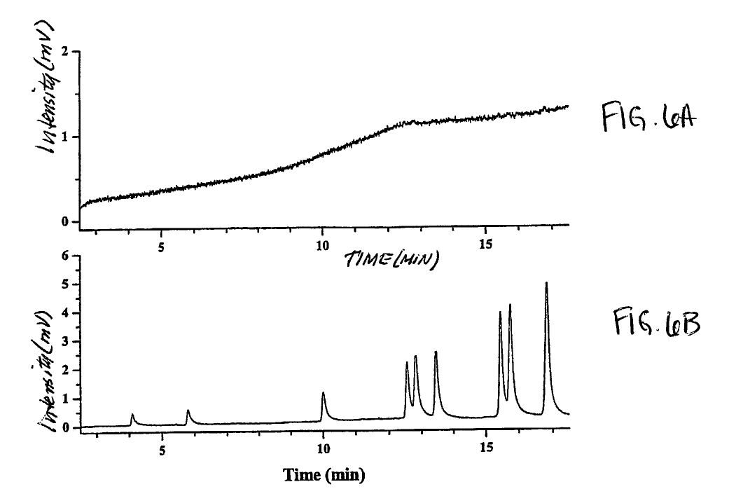

The signal enhancement of the fluorescence detection over the absorbance detection is clearly evident (FIG. 6). As an example of the enhanced detection sensitivity, no peaks were observed in the absorbance chromatogram (FIG. 6A), whereas the peaks were clearly visible and identifiable in the fluorescence chromatogram (FIG. 6B).

### Example 4

### Use of Intercalating Dyes in DHPLC

DHPLC analyses were performed using a Transgenomic Model 3500HT WAVE® nucleic acid fragment analysis system. The system consisted of an Hitachi D-7000 interface, Hitachi D-7100 pump, Hitachi D-7250 autosampler, Hitachi D-7300 column heater with stainless preheat, Hitachi D-7400 UV detector, set at 260 nm, ERC-345a vacuum degasser module, and an Intel Pentium computer including Hitachi HSM control and acquisition software and WAVEMAKER® v. 4.1.38 software (Transgenomic). The aqueous mobile phase consisted of Buffer A: 100 mM triethylammonium acetate (TEAA) (Transgenomic), and Buffer B: 100 mM TEM in 25% acetonitrile (Transgenomic). High purity water used for preparing buffer solutions was obtained using a Milli-Q water system (Millipore, Milford, Mass.). The buffers can be made to an all gravimetric formulation i.e. all components can be weighed out), and can be prepared under temperature controlled conditions (e.g. in a water bath).

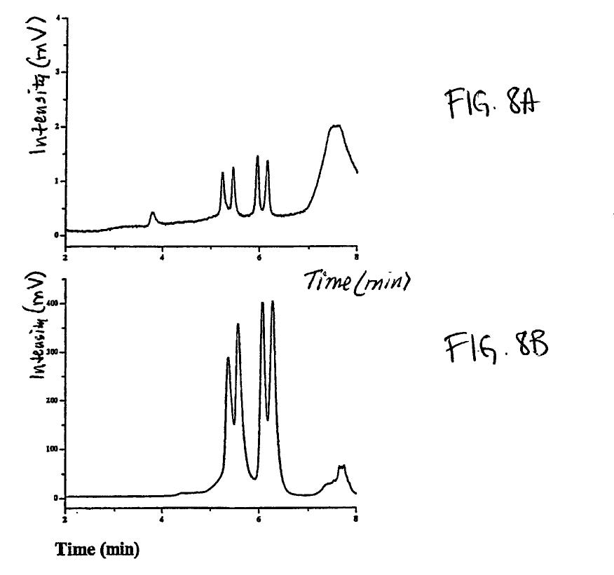

A DYS271 mutation standard (part no. 560077, Transgenomic) was analyzed as follows. The injection volume was 2 μl. The mobile phase flow rate was 0.9 ml/min. The intercalating dye solution included 50 μl CYBR Green 1 dye reagent (Molecular Probes) diluted into 1 liter of water. The flow rate for the dye solution was 0.9 ml/min. The column temperature was 50° C. for the analysis shown in FIG. 7, and was 56° C. (partially denaturing conditions) for FIG. 8.

In FIGS. 7 and 8, the separation column (4.6 mm ID×50 mm) contained alkylated poly(styrene-divinylbenzene) beads (DNASep® column, Transgenomic). The column was eluted at a flow rate of 0.9 ml/min, with the following gradient:

| Time | % B |
|---|---|
| 0.0 | 46 |
| 0.5 | 51 |
| 5.0 | 60 |
| 5.1 | 100 |
| 5.6 | 100 |
| 5.7 | 46 |
| 8.0 | 46 |

The DYS271 Mutation Standard contained equal amounts of the double stranded sequence variants 168A and 168G of the 209 base pair fragment from the human Y chromosome locus DYS271 (GenBank accession Number S76940). The A→G transition position 168 in the sequence was reported by Seielstad et al. (Human Molecular Genetics 3:2159-2161 (1994)) and the preparation of the variants has been described (Narayanaswami et al, Genetic Testing 5:9-16 (2001)). The following is the sequence of the 168A variant:

| | |
|---|---|
| AGGCACTGGTCAGAATGAAGTGAATGGCACACAGGACAAGTCCAGACCCAGGA | (SEQ ID NO:1) |
| AGGTCCAGTAACATGGGAGAAGAACGGAAGGAGTTCTAAAATTCAGGGCTCCCTTGG | |
| GCTCCCCTGTTTAAAAATGTAGGTTTTATTATTATATTTCATTGTTAACAAAAGTCCATG | |
| AGATCTGTGGAGGATAAAGGGGGAGCTGTATTTTCCATT | |

In the standard, the variants are present at a DNA concentration of 45 μg/mL and suspended in 10 mM Tris-HCl, pH 8, 1 mM EDTA.

Prior to DHPLC analysis, the sample was subjected to the following hybridization procedure: denaturation at 95° C. for 12 minutes, followed by slow cooling to 25° C. over a 30 min period.

A UV detector was located downstream of the column, followed in series by a post-column reactor tee, followed by a fluorescence detector. FIGS. 7A and 8A show the absorbance at A260. FIGS. 7B and 8B show the sample as analyzed by fluorescence detection and demonstrate the increase in sensitivity when intercalating dyes were used in conjunction with a fluorescence detector. The signal enhancement was about 580-fold for the fluorescence signal as compared to the UV absorbance signal.

While the foregoing has presented specific embodiments of the present invention, it is to be understood that these embodiments have been presented by way of example only. It is expected that others will perceive and practice variations which, though differing from the foregoing, do not depart from the spirit and scope of the invention as described and claimed herein.

All patent applications, patents, and literature references cited in this specification are hereby incorporated by reference in their entirety. In case of conflict or inconsistency, the present description, including definitions, will control.

The invention claimed is:

1. A method for analyzing one or more polynucleotides in a mixture, said method comprising:
a) separating said polynucleotides using a liquid chromatographic separation device wherein said polynucleotides are eluted from said device;
b) contacting eluted polynucleotides with intercalating dye such that said dye binds to said eluted polynucleotides; and
c) detecting said dye bound to said eluted polynucleotides.

2. The method of claim 1 wherein said device comprises a reverse phase separation column.

3. The method of claim 1 wherein said device comprises an ion exchange column.

4. The method of claim 1 wherein said contacting further includes flowing said mixture through a post-column reactor.

5. The method of claim 4 wherein said reactor is a mixing tee or mixing cross.

6. The method of claim 1 wherein said dye comprises a fluorescent dye.

7. The method of claim 1 wherein said method further includes heating said reagent such that said column and said reagent are retained at essentially the same temperature.

8. The method of claim 1 wherein said polynucleotides include at least one of single stranded and double stranded molecules.

9. The method of claim 1 wherein said polynucleotides comprise DNA.

10. The method of claim 1 wherein said polynucleotides comprise RNA.

11. The method of claim 1 wherein said polynucleotides comprise homoduplex and heteroduplex molecules.

12. The method of claim 1 wherein said dye comprises a nucleic acid stain.

13. The method of claim 1 wherein said dye is selected from the group consisting of SYBR Green I, SYBR Green II, and mixtures thereof.

14. The method of claim 1 wherein said dye comprises SYBR Gold nucleic acid stain.

15. The method of claim 1 further including analyzing polynucleotides of step (b) by mass spectral analysis.

16. A composition comprising the polynucleotide product of claim 1.

17. A method for analyzing one or more polynucleotides, said method comprising:
a) a step for separating said polynucleotides using a liquid chromatographic separation device wherein said polynucleotides are eluted from said device;
b) a step for contacting eluted polynucleotides with intercalating dye such that said dye binds to said eluted polynucleotides; and
c) a step for detecting said dye bound to said eluted polynucleotides.

18. An apparatus for analyzing polynucleotides comprising:
a) a liquid chromatographic separation column capable of separating polynucleotides by ion-pair reverse-phase high performance liquid chromatography; and
b) a reactor for mixing intercalating dye reagent with polynucleotides eluted from said column.

19. The apparatus of claim 18 wherein said column comprises a reverse phase separation column.

20. The apparatus of claim 18 wherein said column comprises an ion exchange column.

21. The apparatus of claim 18 further including a detector capable of detecting said dye bound to polynucleotides.

22. The apparatus of claim 21 wherein said detector comprises a fluorescence detector.

23. The apparatus of claim 18 wherein said reactor comprises a mixing tee.

24. The apparatus of claim 18 further including a heater for heating said dye reagent to essentially the same temperature as said column.

25. The apparatus of claim 18 including an ultraviolet detector.

26. The apparatus of claim 18 including a mass spectrometer operatively coupled to said column.

27. The apparatus of claim 18 further including:
c) conduit connected to the end of said column for conducting mobile phase eluting from said column, said reactor connected to said tubing;
d) a reservoir containing said intercalating dye reagent including conduit for operatively connecting said reservoir to said reactor; and
e) a pump for pumping said dye reagent into said reactor such that said dye reagent mixes with said mobile phase.

28. An apparatus for analyzing polynucleotides comprising:
a) a chromatographic means for separating one or more polynucleotides; and
b) means for mixing intercalating dye with polynucleotides eluted from said device.

29. The apparatus of claim 28 wherein said chromatographic means comprises a reverse phase separation column.

30. The apparatus of claim 28 wherein said chromatographic means comprises an ion exchange column.

31. The apparatus of claim 28 including means for detecting intercalating dye bound to polynucleotides eluted from said chromatographic means.

32. The apparatus of claim 31 wherein said detector means comprises a fluorescence detector.

33. The apparatus of claim 28 wherein said means for mixing comprises a post-column reactor.

34. The apparatus of claim 33 wherein said means for mixing comprises a mixing tee adapted to mix said intercalating dye and polynucleotides eluted from said means for separating.

35. A chromatographic method for separating heteroduplex and homoduplex DNA molecules in a mixture, the method comprising:
applying the mixture to a stationary reverse phase support,
eluting the heteroduplex and homoduplex molecules of said mixture with a mobile phase containing an ion-pairing reagent and an organic solvent, where said eluting is carried out under conditions effective to at least partially denature said heteroduplexes and where said eluting results in the separation of said heteroduplexes from said homoduplexes,
contacting the heteroduplex and homoduplex molecules with intercalating dye reagent after said eluting, and
detecting said dye bound to said heteroduplex and homoduplex molecules.

36. The method of claim 35 wherein the stationary support is composed of an alkylated base material, said base material selected from the group consisting of silica, alumina, zirconia, polystyrene, polyacrylamide, and styrene-divinyl copolymers.

37. The method of claim 35 wherein the mobile phase contains an ion-pairing agent selected from the group consisting of lower alkyl primary, secondary, and tertiary amines, lower trialkylammonium salts and lower quaternary ammonium salts.

38. The method of claim 35 wherein the mobile phase comprises triethylammoniumacetate.

39. The method of claim 34 wherein the mobile phase contains an organic solvent selected from the group consisting of methanol, ethanol, acetonitrile, ethyl acetate, and 2-propanol.

40. The method of claim 35 wherein the mobile phase contains less than about 40% by volume of said organic solvent.

41. The method of claim 35 wherein said method includes heating said reagent to essentially the same temperature as said heteroduplex and homoduplex molecules under said conditions.

42. The method of claim 35 wherein said dye is selected from the group consisting of SYBR Green I stain, SYBR Green II stain, and mixtures thereof.

43. The method of claim 35 wherein said dye comprises SYBR Gold nucleic acid stain.

44. A kit for detecting polynucleotides, said kit comprising:
a) intercalating dye reagent; and
b) a reactor for mixing intercalating dye reagent with mobile phase eluting from a liquid chromatography column.

45. The kit of claim 44 further comprising a liquid chromatography column.

46. The kit of claim 44 further comprising a pump for pumping a solution of said dye into said reactor.

47. The kit of claim 44 further comprising a detector for detecting said dye.

48. The kit of claim 44 further comprising conduit for connecting said reactor to said column.

49. The kit of claim 44 further comprising a standard mixture of polynucleotides.

50. The kit of claim 49 wherein said standard mixture of polynucleotides comprises double stranded DNA.

51. The kit of claim 49 where said standard mixture of polynucleotides comprises a mutation standard.

52. The kit of claim 49 wherein said standard mixture of polynucleotides comprises double-stranded polynucleotides.

53. The kit of claim 44 wherein said dye reagent comprises a nucleic acid stain.

54. The kit of claim 44 wherein said dye reagent is selected from the group consisting of SYBR Green I stain, SYBR Green II stain, and mixtures thereof.

55. The kit of claim 44 wherein said dye reagent comprises SYBR Gold nucleic acid stain.

56. An apparatus for analyzing polynucleotides comprising:
a) means for chromatographic separation wherein one or more polynucleotides can be applied to said means for chromatographic separation and can be eluted from said means for chromatographic separation;
b) means for adding or mixing intercalating dye with polynucleotides eluted from said means for chromatographic separation; and
c) means for detecting intercalating dye bound to polynucleotides eluted from said means for chromatographic separation.

57. The apparatus of claim 56 wherein said means for chromatographic separation comprises a reverse phase liquid chromatography column.

58. The apparatus of claim 56 wherein said means for adding or mixing comprises a mixing tee, a liquid flow-through reactor, or a hollow fiber membrane.

59. An apparatus for analyzing polynucleotides comprising:
i) a liquid chromatographic column having an outlet;
ii) a mixing tee having a first inlet, a second inlet, and an outlet with the first inlet in fluid communication with the outlet of the chromatographic column;
iii) wherein the second inlet is in fluid communication with a fluid source, wherein said fluid source comprises an intercalating dye reagent.

60. The apparatus of claim 59 further include a heater for heating said dye reagent.

61. A liquid chromatographic apparatus comprising a silica based chromatographic column means or a polymeric based chromatographic column means, a reservoir of mobile phase in fluid communication with said column means, a chromatographic pump means to add the mobile phase to the column means, whereby the sample comprising a mixture of at least one polynucleotide is eluted through the column means, and component species of said mixture appear in chromatographically displaced form in the effluent of the chromatographic column means, and further including a post-column reactor means through which the effluent of the chromatographic column means is fed to a liquid chromatographic detector, a medium comprising intercalating dye reagent, said reactor means being in operative contact or communication with said medium for transfer of the reagent into the effluent of the chromatographic column means.

62. The apparatus of claim 61 wherein said post-column reactor means is selected from the group consisting of a hollow fiber membrane, a mixing tee, and a mixing cross.

63. The apparatus of claim 61 including a pump for pumping said medium into effluent from said chromatographic column means.

64. The apparatus of claim 63 wherein said pump is a syringe, a peristaltic pump, or an HPLC pump.

65. A chromatographic apparatus for separating polynucleotides, said apparatus comprising:
a reverse phase separation column,
a post-column reactor located downstream of said column,
a medium containing intercalating dye, wherein said reactor is adapted to mix mobile phase eluted from said column with said medium,
a fluorescence detector downstream of said reactor for detecting intercalating dye bound to polynucleotides.

66. The apparatus of claim 65 wherein said column comprises silica stationary support.

67. The apparatus of claim 65 wherein said column comprises polymeric stationary support.

68. A method for analyzing one or more polynucleotides, said method comprising:
a) separating said polynucleotides using capillary electrophoresis;
b) contacting said polynucleotides with intercalating dye; and
c) detecting said dye bound to said polynucleotide.
